(19) United States
(12) Patent Application Publication
(10) Pub. No.: US 2019/0328854 A1
(43) Pub. Date: Oct. 31, 2019

(54) C5 IMMUNIZATION FOR AUTOLOGOUS ANTI-C5 ANTIBODY PRODUCTION

(71) Applicants: The Cleveland Clinic Foundation, Cleveland, OH (US); GEORGIA TECH RESEARCH CORPORATION, Atlanta, GA (US)

(72) Inventors: Feng Lin, Cleveland, OH (US); M.G. Finn, Atlanta, GA (US)

(21) Appl. No.: 16/328,197

(22) PCT Filed: Aug. 29, 2017
(86) PCT No.: PCT/US17/49059

§ 371 Date: Feb. 25, 2019

Related U.S. Application Data
(60) Provisional application No. 62/380,824, filed on Aug. 29, 2016.

(51) Int. Cl. A61K 39/00; A61P 37/06; C12N 15/86; C07K 14/47

(52) U.S. Cl. CPC A61K 39/0008; A61P 37/06; C12N 15/86; C07K 14/472; A61K 2039/5258; C12N 2795/18141; A61K 2039/605; A61K 2039/6075; A61K 2039/64; C12N 2795/18123; C12N 2795/18134; A61K 2039/55566

(57) ABSTRACT

Provided herein are immunogenic compositions comprising an immunogen with at least one complement component 5 (C5) epitope, wherein the immunogen is capable of generating autologous anti-C5 antibodies in a subject. In certain embodiments, such compositions are employed for treating and preventing complement component 5 (C5) related diseases. In certain embodiments, the immunogenic compositions comprise virus like particles and/or PADRE sequences, in addition to the C5 epitope(s).

The present application claims priority to U.S. Provisional application Ser. No. 62/380,824 filed Aug. 29, 2016, which is herein incorporated by reference in its entirety.

This invention was made with government support under grant numbers DK103581, AR061564, and GM101421, awarded by the National Institutes of Health. The government has certain rights in the invention.

### FIELD OF THE INVENTION

Provided herein are immunogenic compositions comprising an immunogen with at least one complement component 5 (C5) epitope, wherein the immunogen is capable of generating autologous anti-C5 antibodies in a subject. In certain embodiments, such compositions are employed for treating and preventing complement component 5 (C5) related diseases. In certain embodiments, the immunogenic compositions comprise virus like particles and/or PADRE sequences, in addition to the C5 epitope(s).

### BACKGROUND OF THE INVENTION

The complement system is an important part of the innate immune system, being composed of multiple proteins present at varying levels in the blood¹. When complement is activated through any of three general pathways (by antibody-antigen complexes through the classical pathway, by certain carbohydrates through the lectin pathway, or spontaneously through the alternative pathway), C3 is activated to form C5 convertase and cleaves C5 into C5b and C5a. When C5 is activated in this way, membrane attack complexes (MACs) composed of C5b-9 are assembled and form pores through the cell membrane, resulting in lysis or damage of the target cell. At the same time, the small fragment C5a is released into the fluid phase and binds to its receptor (C5aR) on nearby immune cells to promote inflammatory reactions. Activated complement cannot distinguish self from foreign cells, but self-cells express cell surface complement inhibitors, including CD55 and CD59, which confer protection from complement-mediated attack¹. Aberrant complement activation is integrally involved in many diseases, and many complement components are currently being tested as new targets for drug development². C5 is a promising example because its concentration in blood is relatively low (˜100 μg/mL for C5 v.s. ˜1500 μg/mL for C3), and C5 inhibition should theoretically inhibit both the formation of MACs, which damage tissue cells, and suppress the release of C5a, a potent inflammation initiator that is involved in many pathological conditions.

Indeed, ECULIZUMAB, a humanized anti-human C5 monoclonal antibody (mAb) has been successfully employed to treat complement-mediated diseases including paroxysmal nocturnal hemoglobinuria (PNH)³ and atypical hemolytic uremic syndrome (aHUS)⁴. In most patients with PNH, inactivation of the gene for phosphatidylinositol glycan A in the glycosyl phosphatidylinositol (GPI) pathway in hematopoietic stem cells results in red blood cells (RBCs) lacking the GPI-anchored cell surface complement inhibitors CD55 and CD59, rendering these cells highly susceptible to complement-mediated lysis⁵. ECULIZUMAB binds to C5 and inhibits the formation of MACs, thereby preventing complement-mediated hemolysis in PNH. Although highly effective, ECULIZUMAB is the most expensive drug on the market with an annual cost of more than $400,000 USD/patient, and in most of the patients, life-long drug administration is required^{6,7}.

### SUMMARY OF THE INVENTION

Provided herein are immunogenic compositions comprising an immunogen with at least one complement component 5 (C5) epitope, wherein the immunogen is capable of generating autologous anti-C5 antibodies in a subject. In certain embodiments, such compositions are employed for treating and preventing complement component 5 (C5) related diseases. In certain embodiments, the immunogenic compositions comprise virus like particles and/or PADRE sequences, in addition to the C5 epitope(s). In particular embodiments, the C5 epitope comprises a synthetic peptide (e.g., recombinantly produced peptide).

In some embodiments, provided herein are composition comprising an immunogen, or a nucleic acid sequence encoding the immunogen (or multiple nucleic acid sequences that combine to form said immunogen), wherein the immunogen comprises at least one complement component 5 (C5) epitope (e.g., 1, 2, 3, 4 . . . 10 . . . 15 . . . 20 or more C5 epitopes which are the same or different).

In particular embodiments, the C5 epitope is a C5b epitope. In other embodiments, the immunogen further comprises a PADRE sequence. In particular embodiments, the immunogen further comprises a virus coat protein (e.g., from Qβ, adeno-virus, HCV, etc.). In particular embodiments, the C5 epitope is formed by an amino acid sequence comprising, consisting essentially of, or consisting of SEQ ID NOS:3-76, or an amino acid sequence with at least 90%-95% sequence identity to SEQ ID NOS:3-76. In other embodiments, the C5 epitope(s) is the only C5 derived sequence in the immunogen, and wherein the amino acid sequence is between 10 and 30 amino acids in length (e.g., 10 . . . 14 . . . 20 . . . 25 . . . 28 . . . 30 amino acids in length). In additional embodiments, the C5 epitope is the only C5 derived sequence in the immunogen, and wherein the amino acid sequence is between 12 and 25 amino acids in length. In other embodiments, the immunogen further comprises a virus like particle. In additional embodiments, the at least one C5 epitope is at least two C5 epitopes. In additional embodiments, the at least one C5 epitope is a least 10 C5 epitopes (e.g., that are all the same, or different).

In some embodiments, the compositions herein further comprise an immune-effective amount of an immunostimulant. In other embodiments, the immunostimulant comprises an adjuvant selected from the group consisting of: Freunds adjuvant, alum, aluminum hydroxide, aluminum phosphate, and calcium phosphate hydroxide. In particular embodiments, the C5 epitope is formed by an amino acid sequence comprising, consisting essentially of, or consisting of SEQ ID NOS:40-76.

In particular embodiments, provided herein is a host cell containing immunogens disclosed herein and/or a nucleic acid sequence encoding such immunogen. In other embodiments, provided herein are compositions comprising a nanoparticle or virus like particle, wherein the nanoparticle or the virus like particle comprises multiple copies of a first peptide arrayed thereon, wherein the first peptide comprises at least one complement component 5 (C5) epitope.

In some embodiments, provided herein are methods of immunizing or vaccinating a subject, comprising delivering any of the immunogen containing compositions described herein. In certain embodiments, the subject has one or more symptoms of a disease selected from: complement mediated hemolysis, tissue damage, paroxysmal nocturnal hemoglobinuria (PNH), atypical hemolytic uremic syndrome (aHUS), Relapsing Neuromyelitis Optica (NMO), Shiga-toxin producing E. coli hemolytic uremic syndrome (STEC-HUS), Delayed Graft Function (DGF), Refractory Generalized Myasthenia Gravis (MG), Membranoproliferative glomerulonephritis (MPGN), Dense-deposit disease (DDD), Cold agglutinin disease, and Catastrophic antiphospholipid syndrome (CAPS). In further embodiments, the immunogenic composition is delivered intravenously, parenterally, or mucosally. In further embodiments, the subject has a human complement component 5 (C5) related disease.

In certain embodiments, provided herein are methods of immunizing a subject (e.g., mouse, human, farm animal, dog, cat, etc.) comprising: administering a composition to a subject, wherein the composition comprises an immunogen, or a nucleic acid sequence(s) encoding the immunogen, wherein the immunogen comprises at least one complement component 5 (C5) epitope, and wherein the administering causes autologous anti-C5 antibodies to be generated in the subject. In some embodiments, the subject is affected with a human complement component 5 (C5) related disease. In further embodiments, the immunogen further comprises a PADRE sequence. In other embodiments, the immunogen further comprises a virus coat protein. In certain embodiments, the C5 epitope is formed by an amino acid sequence comprising, consisting essentially of, or consisting of SEQ ID NOS:3-76, or an amino acid sequence with at least 90% sequence identity to SEQ ID NOS:3-76.

### DESCRIPTION OF THE FIGURES

FIG. 1. Immunization of mice with purified human C5 (hC5) generates anti-hC5 IgGs that do not reduce mouse C5 (mC5) activity. WT C57BL/6 mice were immunized with purified hC5 in CFA or CFA alone (control) and boosted 2 weeks later. One week after the boost, serum samples were collected. (A) serum samples were tested for anti-hC5 IgG antibody titers using a hC5 ELISA, showing high titers of anti-hC5 antibodies developed in the hC5 immunized (IM) mice (detectable at a 1:32000 dilution), but not control mice (not detectable even at a 1:500 dilution). (B) C5 activity in the serum samples were assessed by a modified E^{shA} hemolysis assay and no significant difference was found between the hC5-immunized mice and the control mice. n=5 in each group.

FIG. 2. Design, expression, and purification of the recombinant mouse C5 vaccine. (A) Amino acid sequence of the recombinant C5 vaccine (Opt), composed of 12 computer-identified mouse C5 surface epitopes (non-highlighted amino acids), three copies of the PADRE peptide (highlighted), and a 6×His-tag (highlighted). (B) Reversed-phase HPLC to further purify the recombinant C5P after affinity chromatography. (C) SDS-PAGE analysis of the purified recombinant C5 vaccine. M=markers; lys=whole E. coli lysate; ID=inclusion bodies; C5₁=60 μg of purified recombinant C5 vaccine; C5₂=20 μg of purified recombinant C5 vaccine.

FIG. 3. The recombinant mouse C5 vaccine reduces mouse C5 (mC5) levels and activity in the blood and significantly reduces hemolysis in these mice in a model of PNH. (A) After the last immunization of the recombinant mouse C5 vaccine or CFA alone, serum samples were collected and tested for their anti-mC5 vaccine IgG titers by ELISA (n=4 in each group). (B) Mouse C5 activity in these samples were compared using the modified EshA-based hemolytic assay (n=12 in each group). (C) The recombinant C5 vaccine-immunized mice and control mice were intravenously injected with 200 μL of C5-deficient human serum per mouse, then, 1 h later, were bled retro-orbitally using a heparinized capillary tube, and in vivo intravascular hemolysis was assessed by measuring the OD414 of the plasma samples. (n=4 in each group). IM, immunized.

FIG. 4. Verification of the P2 peptide (SEQ ID NO:4) on the surface of mouse C5 protein by homology modeling. A three-dimensional model of mouse C5 was constructed based on the published human C5 X-ray crystal structure. Mouse C5 protein shares 89% sequence homology with human C5 protein. The P2 epitope (red/dark shading) was found to be exposed on mouse C5 surface based on this model. (A) Ribbon representation of the mouse C5 structure model. Amino (N)- and carboxy (C)-terminus of the protein are also labeled. (B) Surface representation of the mouse C5 structure model.

FIG. 5. Expression and characterization of hybrid Qβ VLPs. A) Schematic representation of plasmids used for particle expression. PADRE sequence highlighted in gold; C5 P2 peptide highlighted in blue. CP: coat protein. B) Electrophoretic analysis of VLPs displaying the P2 peptide. Bands for the wild-type CP and the CP-P2 fusion are indicated. Particle composition calculated using densitometry. C) FPLC elution profile for Qβ-P2 VLPs showing particles are intact and greater than 95% pure. D) Dynamic light scattering histogram of Qβ-P2 VLPs showing particles are monodisperse in size with a hydrodynamic radius of 16.9 nm.

FIG. 6. The VLP-C5 vaccine elicited high titers of anti-C5 autoantibodies and significantly decreased mouse C5 hemolytic activity. WT mice were immunized subcutaneously with the VLP-C5 vaccine or empty VLP as controls, and boosted once or twice after every other week. IgG titers of the anti-recombinant mouse C5 in the sera were measured before the immunization and after the last boost. (A) Before immunization, anti-C5 antibodies were not detectable in both two groups of mice. After the last boost, anti-mouse C5 IgGs were highly detectable in sera from VLP-C5 vaccine immunized mice but not in sera from the control mice. (B). In the modified ex vivo hemolysis assays, sera from the same VLP-C5 vaccine immunized mice and control mice were incubated with antibody-sensitized sheep RBCs (E^{shA}) to quantitate mouse C5 activity. Sera from C5 KO mice were used as negative controls. n=13 in each group.

FIG. 7. VLP-C5 vaccine immunization protects mice from complement-mediated hemolysis and hemoglobinuria in a model of PNH. (A) VLP-C5 vaccine immunized mice and empty VLP-immunized control mice were injected 200 μL of human C5-depleted serum intraperitoneally. 1 hour later, blood was collected through venous sinus of the eye and the resultant sera were diluted at a ratio of 1:10 using PBS before OD values were measured at 414 nm. n=8 in each group. (B) Urea samples were collected from the VLP-C5 vaccine immunized mice and empty VLP-immunized control mice, and hemoglobinuria were measured by reading OD₄₁₄ after 1:10 dilution with PBS. n=5 in each group.

FIG. 8. VLPs displaying P2, P3 or P12 elicit mouse anti mouse C5 antibodies. Sera collected from mice without immunization (wo immunization), mice immunized with VLPs displaying different C5 epitopes (P2, P3 or P12) or the control empty VLPs (Ctrl VLP) were assessed for mouse C5-reactive IgG levels by conventional ELISA.

FIG. 9. Immunization of VLPs displaying P2 or P3 reduces blood C5 activities ex vivo. Sera collected from C5 deficient mice (C5KO), mice without immunization (WT), mice immunized with VLPs displaying different C5 epitopes (P2, P3 or P12) or the control empty VLPs (Ctrl VLP) were assessed for C5 activity using a modified sheep RBC-based C5 activity assay.

### DEFINITIONS

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. The peptides in SEQ ID NOS:3-76 represent immunogens of the present invention.

As used herein the term “epitope” refers to a peptide sequence which elicits an immune response, from either T cells or B cells or antibody

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. In certain embodiments, provided herein are vectors comprising nucleic acid that encode the immunogens (e.g., SEQ ID NOS:3-76) described herein.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.).

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

The term “nanoparticle” as used herein refers to a small particle used to array immunogens which may be comprised of protein, lipid, carbohydrate or combination thereof or may be a “virus like particle” which mimics a virus in structure but lacks replicative capability.

As used herein an “immunostimulant” may refer to an adjuvant, including but not limited to Freunds adjuvant, inorganic compounds (e.g., alum, aluminum hydroxide, aluminum phosphate, calcium phosphate hydroxide), mineral oil (e.g., paraffin oil), bacterial products (e.g., killed bacteria, Bordetella pertussis, Mycobacterium bovis, toxoids), nonbacterial organics (e.g., squalene, thimerosal), detergents (e.g., Quil A), plant saponins from quillaja, soybean, polygala senega, cytokines (e.g., IL-1, IL-2, IL-12), and food Based oil (e.g., adjuvant 65). The immunogenic compositions described herein may further comprise one or more immunostimulants.

### DESCRIPTION OF THE INVENTION

Provided herein are immunogenic compositions comprising an immunogen with at least one complement component 5 (C5) epitope, wherein the immunogen is capable of generating autologous anti-C5 antibodies in a subject. In certain embodiments, such compositions are employed for treating and preventing complement component 5 (C5) related diseases. In certain embodiments, the immunogenic compositions comprise virus like particles and/or PADRE sequences, in addition to the C5 epitope(s).

The complement system is emerging as a new target for treating many diseases. For example, ECULIZUMAB, a humanized monoclonal antibody against complement component 5 (C5), has been approved for paroxysmal nocturnal hemoglobinuria (PNH) in which patient erythrocytes are lysed by complement. In work conducted during the development of the present invention, we developed vaccines to elicit autologous anti-C5 antibody production in mice for complement inhibition. Immunization of mice with a conservative C5 xenoprotein raised high titers of IgGs against the xenogenous C5, but these antibodies did not reduce C5 activity in the blood. In contrast, an autologous mouse C5 vaccine containing multiple predicted epitopes together with a tolerance-breaking peptide was found to induce anti-C5 autoantibody production in vivo, resulting in decreased hemolytic activity in the blood. We further validated a peptide epitope within this C5 vaccine and created recombinant virus-like particles (VLPs) displaying this epitope fused with the tolerance breaking peptide. Immunizing mice with these nanoparticles elicited strong humoral responses against recombinant mouse C5, reduced hemolytic activity, and protected the mice from complement-mediated intravascular hemolysis in a model of PNH. This work demonstrated that autologous C5-based vaccines could be an effective alternative or supplement for treating complement-mediated diseases such as PNH.

In certain embodiments, the peptide employed as the immunogen comprises, consists essentially of, or consists of a peptide shown in Tables 3 or 4, or a peptide with at least 90% or 95% sequence identity with one of these peptides. In Table 3, P1-P12 represent murine derived C5 peptides, while peptides P1-H-P12-H are human derived C5 peptides.

TABLE 3

| Peptide | | SEQ ID |
|---|---|---|
| Name | Sequence | NO: |
| P1 | ISADSRKEKACKPE | 3 |
| P1-H | ISAETRKQTACKPE | 40 |
| P2 | ASYKPSKEESTSGS | 4 |
| P2-H | ASYKPSREESSSGS | 41 |
| P3 | QETSDLETKRSITH | 5 |
| P3-H | QETSDLDPSKSVTR | 42 |
| P4 | KCQLENGSFKENSQ | 6 |
| P4-H | NYQLDNGSFKENSQ | 43 |
| P5 | NANADDSHYRDDS | 7 |
| P5-H | NANADDSQENDEP | 44 |
| P6 | YSLGDDLKPAKRET | 8 |
| P6-H | YSLNDDLKPAKRET | 45 |
| P7 | LPEENQASKEYEAV | 9 |
| P7-H | LPEENQAREGYRAI | 46 |
| P8 | KTGEAADENSEVTF | 10 |
| P8-H | KTGEAVAEKDSEITF | 47 |
| P9 | RVVPEGVKRESYAG | 11 |
| P9-H | RVVPEGVKRESYSG | 48 |
| P10 | KSDLGCGAGGGHDN | 12 |
| P10-H | KSDLGCGAGGGLNN | 49 |
| P11 | HTDKPVYTPDQSVK | 13 |
| P11-H | HTDKPVYTPDQSV | 50 |
| P12 | DTLKRPDSSVPSSG | 14 |
| P12-H | DNLQHKDSSVPNTG | 52 |

Table 4 includes truncated (on the left side of the table) and extended (right side of table) sequences of the P1-H-P-12-H sequences.

TABLE 4

| | SEQ ID |
|---|---|
| Sequence | NO: |
| SAETRKQTACKP | 53 |
| DLTISAETRKQTACKPEIAY | 54 |
| SYKPSREESSSG | 55 |
| VACASYKPSREESSSGSSHA | 56 |
| ETSDLDPSKSVT | 57 |
| DVNQETSDLDPSKSVTRVDD | 58 |
| YQLDNGSFKENS | 59 |
| LVENYQLDNGSFKENSQYQP | 60 |
| ANADDSQENDE | 61 |
| FLTNANADDSQENDEPCKE | 62 |
| SLNDDLKPAKRE | 63 |
| VRVYSLNDDLKPAKRETVLT | 64 |
| PEENQAREGYRA | 65 |
| APDLPEENQAREGYRAIAYS | 66 |
| TGEAVAEKDSEIT | 67 |
| DIYKTGEAVAEKDSEITFIKK | 68 |
| VVPEGVKRESYS | 69 |
| KTLRVVPEGVKRESYSGVTL | 70 |
| SDLGCGAGGGLN | 71 |
| FLEKSDLGCGAGGGLNNANV | 72 |
| TDKPVYTPDQS | 73 |
| LFIHTDKPVYTPDQSVKVRV | 74 |
| NLQHKDSSVPNT | 75 |
| FWKDNLQHKDSSVPNTGTAR | 76 |

In some embodiments, the synthetic peptides that provide the C5 epitopes are from 8-50 (e.g., 9-15 amino acids, 15-45 amino acids) or precisely 9 or 15 amino acids. In some embodiments, synthetic peptides are be assembled as fusion peptides (e.g., a peptide linked to a hapten). In some embodiments the peptides are fused each with an immunoglobulin Fc component or parts thereof by a short peptide linker. In other embodiments the peptides of interest are arrayed on a nanoparticle or other nanovehicle such as a nanosphere, virus like particle, or microparticle. In further embodiments a nucleotide sequence encoding said peptides are delivered to the patient for in vivo expression.

In some embodiments, the immunogens described herein are administered to the patient affected by a C5 mediated disease along with an adjuvant or an immune stimulant or other molecule to induce local inflammation. Peptides as provided by the technology provided herein (e.g., provided in SEQ ID NOS:3-76) find use in compositions that are vaccines, vaccine components, and/or a pharmaceutical comprising a peptide or fusion thereof, DNA/RNA sequences, or expression vectors according to the technology. Where appropriate, this pharmaceutical additionally comprises a pharmaceutically compatible carrier. Suitable carriers and the formulation of such pharmaceuticals are known to a person skilled in the art. Suitable carriers are, e.g., phosphate-buffered common salt solutions, water, emulsions, e.g. oil/water emulsions, wetting agents, sterile solutions, etc. The pharmaceuticals may be administered orally or parenterally. The methods of parenteral administration comprise the topical, intra-arterial, intramuscular, subcutaneous, intramedullary, intrathecal, intraventricular, intravenous, intraperitoneal, or intranasal administration. The suitable dose is determined by the attending physician and depends on different factors, e.g. the patient's age, sex and weight, the kind of administration etc.

In some embodiments, nucleic acids expressing the immunogens herein are present in a host cell in vitro for the production of the vaccine molecule. Recombinant methods for producing polypeptides in a cell culture are well known in the art. For example, in some embodiments, the polypeptides a peptide or fusion thereof are expressed in a bacterial culture such as a culture of E. coli and the polypeptides are purified and isolated from the culture to provide the vaccine. In some embodiments, the host cell is a eukaryotic cell kept in cell culture (e.g., transfected into CHO cells, NSO cells, 293E cells and Cos-7 cells) and may or may not by a transformed cell in some embodiments. In some embodiments, the host cell is an autologous cell from the subject to be treated.

In one embodiment, an immunogen of the present invention is administered parenterally. In another embodiment the immunogen is administered to a mucosal surface such as the nasal cavity, cervix, or other mucosa. In another embodiment, the immunogen is administered orally so as to permit presentation to the buccal or gastrointestinal mucosa. In some forms of oral administration the immunogen is encapsulated in an enteric capsule or gel capsule. In yet other embodiments, the immunogen is combined into a chewable form. In some embodiments, the immunogen molecule can be applied topically to the skin.

In some embodiments, the present invention provides immunogen compositions comprising a C5 epitope as provided herein. The present invention is not limited by the particular formulation of a composition comprising an immunogen. Indeed, a vaccine or immunogen composition of the present invention may comprise one or more different agents in addition to an immunogen. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a immunogen composition comprises an agent or co-factor that enhances the ability of the antigenic C5 unit to induce an immune response (e.g., an adjuvant). In some embodiments, the presence of one or more co-factors or agents reduces the amount of antigenic unit required for induction of an immune response (e.g., a protective immune response (e.g., protective immunization)). In some embodiments, the presence of one or more co-factors or agents is used to skew the immune response towards a cellular (e.g., T-cell mediated) or humoral (e.g., antibody-mediated) immune response. The present invention is not limited by the type of co-factor or agent used in a therapeutic agent of the present invention.

Adjuvants are described in general in Vaccine Design—the Subunit and Adjuvant Approach, edited by Powell and Newman, Plenum Press, New York, 1995, incorporated by reference herein in its entirety for all purposes. The present invention is not limited by the type of adjuvant utilized (e.g., for use in a composition (e.g., a pharmaceutical composition)). For example, in some embodiments, suitable adjuvants include an aluminium salt such as aluminium hydroxide gel (e.g., alum) or aluminium phosphate. In some embodiments, an adjuvant may be a salt of calcium, iron, or zinc, or it may be an insoluble suspension of acylated tyrosine, or acylated sugars, cationically or anionically derivatized polysaccharides, or polyphosphazenes.

In some embodiments, an immunogenic oligonucleotide containing unmethylated CpG dinucleotides (“CpG”) is used as an adjuvant. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al., J. Immunol, 1998, 160(2):870-876; McCluskie and Davis, J. Immunol., 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63), LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., WO93/13202 and WO92/19265, each of which is hereby incorporated by reference), and other immunogenic substances (e.g., that enhance the effectiveness of a composition of the present invention) are used with a composition comprising a peptide or fusion thereof of the present invention.

Additional examples of adjuvants that find use in the present invention include poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Adjuvants may be added to a composition comprising an immunogen, or the adjuvant may be formulated with carriers, for example liposomes or metallic salts (e.g., aluminium salts (e.g., aluminium hydroxide)) prior to combining with or co-administration with a composition. In some embodiments, a composition comprising an immunogen comprises a single adjuvant. In other embodiments, a composition comprises two or more adjuvants (See, e.g., WO 94/00153; WO 95/17210; WO 96/33739; WO 98/56414; WO 99/12565; WO 99/11241; and WO 94/00153, each of which is hereby incorporated by reference in its entirety).

In some embodiments, a composition comprising an immunogen described herein comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives is contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose.

In some embodiments, a composition of the present invention may comprise sterile aqueous preparations. Acceptable vehicles and solvents include, but are not limited to, water, Ringer's solution, phosphate buffered saline, and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, subcutaneous, intramuscular, intraperitoneal, intravenous, or administration via other routes may be found in Remington's Pharmaceutical Sciences, Mack Publishing Company, Easton, Pa.

A composition comprising an immunogen of the present invention can be used therapeutically (e.g., to enhance an immune response) or as a prophylactic (e.g., for immunization (e.g., to prevent signs or symptoms of disease)). A composition comprising a peptide or fusion thereof of the present invention can be administered to a subject via a number of different delivery routes and methods.

In some embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal, pulmonary, vaginal, and rectal techniques), as well as European Publication No. 517,565 and Illum et al., J. Controlled Rel., 1994, 29:133-141 (e.g., for techniques of intranasal administration), each of which is hereby incorporated by reference in its entirety). Alternatively, the compositions of the present invention may be administered dermally or transdermally using standard techniques (See, e.g., Remington: The Science arid Practice of Pharmacy, Mack Publishing Company, Easton, Pa., 19th edition, 1995). The present invention is not limited by the route of administration.

Compositions of the present invention may also be administered via a vaginal route. In such cases, a composition comprising an immunogen described herein may comprise pharmaceutically acceptable excipients and/or emulsifiers, polymers (e.g., CARBOPOL), and other known stabilizers of vaginal creams and suppositories. In some embodiments, compositions of the present invention are administered via a rectal route. In such cases, compositions may comprise excipients and/or waxes and polymers known in the art for forming rectal suppositories.

In some embodiments, the same route of administration (e.g., systemic, or mucosal administration) is chosen for both a priming and boosting vaccination. In some embodiments, multiple routes of administration are utilized (e.g., at the same time, or, alternatively, sequentially) in order to stimulate an immune response.

For example, in some embodiments, a composition comprising an immunogen described herein is administered intravenously or to a mucosal surface of a subject in either a priming or boosting vaccination regime. Alternatively, in some embodiments, the composition is administered systemically in either a priming or boosting vaccination regime.

In some embodiments, a composition comprising an immunogen is administered to a subject in a priming vaccination regimen via mucosal administration and a boosting regimen via systemic administration. In some embodiments, a composition comprising an immunogen is administered to a subject in a priming vaccination regimen via systemic administration and a boosting regimen via mucosal or systemic administration. Examples of systemic routes of administration include, but are not limited to, a parenteral, intramuscular, intradermal, transdermal, subcutaneous, intraperitoneal, or intravenous administration.

Thus, in some embodiments, a composition comprising a peptide or fusion thereof of the present invention may be used to protect and/or treat a subject susceptible to, or suffering from, a disease by means of administering the composition by mucosal, intramuscular, intraperitoneal, intradermal, transdermal, pulmonary, intravenous, subcutaneous or other route of administration described herein. Methods of systemic administration of the vaccine preparations may include conventional syringes and needles, or devices designed for ballistic delivery of solid vaccines (See, e.g., WO 99/27961, hereby incorporated by reference), or needleless pressure liquid jet device (See, e.g., U.S. Pat. Nos. 4,596,556; 5,993,412, each of which are hereby incorporated by reference), or transdermal patches (See, e.g., WO 97/48440; WO 98/28037, each of which are hereby incorporated by reference). The present invention may also be used to enhance a immunogenicity of antigens applied to the skin (transdermal or transcutaneous delivery, See, e.g., WO 98/20734; WO 98/28037, each of which are hereby incorporated by reference). Thus, in some embodiments, the present invention provides a delivery device for systemic administration, pre-filled with the vaccine composition (e.g., peptides with an amino acid sequence selected from SEQ ID Nos:3-76) of the present invention.

The present invention is not limited by the type of subject administered (e.g., in order to stimulate an immune response (e.g., in order to generate protective immunity (e.g., mucosal and/or systemic immunity))) a composition of the present invention. Indeed, a wide variety of subjects are contemplated to be benefited from administration of a composition of the present invention. In preferred embodiments, the subject is a human. In some embodiments, the general public is administered (e.g., vaccinated with) a composition of the present invention (e.g., to prevent the occurrence or spread of disease). For example, in some embodiments, compositions and methods of the present invention are utilized to vaccinate a group of people (e.g., a population of a region, city, state and/or country) for their own health (e.g., to prevent or treat disease).

A composition of the present invention may be formulated for administration by any route, such as mucosal, oral, transdermal, intranasal, parenteral or other route described herein. The compositions may be in any one or more different forms including, but not limited to, tablets, capsules, powders, granules, lozenges, foams, creams or liquid preparations.

Topical formulations of the present invention may be presented as, for instance, ointments, creams or lotions, foams, and aerosols, and may contain appropriate conventional additives such as preservatives, solvents (e.g., to assist penetration), and emollients in ointments and creams. Topical formulations may also include agents that enhance penetration of the active ingredients through the skin. Exemplary agents include a binary combination of N-(hydroxyethyl) pyrrolidone and a cell-envelope disordering compound, a sugar ester in combination with a sulfoxide or phosphine oxide, and sucrose monooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like. Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a vaccine composition of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of a nanomulsion and antigenic unit present in the concentrated composition. Concentrated compositions are contemplated to be useful in a setting in which large numbers of subjects may be administered a composition of the present invention (e.g., an clinic, hospital, school, etc.). In some embodiments, a composition comprising a peptide or fusion thereof of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks. In some embodiments, following an initial administration of a composition of the present invention (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration. The dosage regimen will also, at least in part, be determined by the need of the subject and be dependent on the judgment of a practitioner. Dosage units may be proportionately increased or decreased based on several factors including, but not limited to, the weight, age, and health status of the subject. In addition, dosage units may be increased or decreased for subsequent administrations (e.g., boost administrations).

The present invention further provides kits comprising the vaccine compositions comprised herein. In some embodiments, the kit includes all of the components necessary, sufficient or useful for administering the vaccine. For example, in some embodiments, the kits comprise devices for administering the vaccine (e.g., needles or other injection devices), temperature control components (e.g., refrigeration or other cooling components), sanitation components (e.g., alcohol swabs for sanitizing the site of injection) and instructions for administering the vaccine.

### EXAMPLES

### Example 1

This Examples describes the development of methods and compositions for eliciting a moderate immune response against C5. Such methods and compositions could be used as a cost-effective treatment of diseases such as PNH caused by excess complement activity. In this Example virus-like particles (VLPs) were employed as the platform due to their immunogenic nature, well-defined structure, ability to present a wide variety of potential epitopes, and ease of production. While VLPs resemble viruses in most ways relevant to the generation of an immune response (repetitive structure, lymphatic trafficking, B cell recognition, T cell stimulation, packaged bacterial RNA)^{8,9}, they are non-infectious and can act as self-adjuvants capable of breaking immune tolerance¹⁰⁻¹². The bacteriophage Qβ is a highly effective and easily-modified VLP platform for these purposes¹³. It has been reported that both the direct attachment of antigens to the surface of Qβ VLPs using chemical crosslinkers and the production of recombinant Qβ VLPs that display antigen epitopes on their surface using gene engineering techniques are effective in eliciting strong immune responses against various antigens for vaccine development. Such Qβ VLP-based vaccines have been found to be safe in multiple Phase I and II clinical trials¹⁴⁻¹⁸ (all of which are herein incorporated by reference in their entireties).

To explore the feasibility of eliciting anti-C5 antibody production from the host immune system to prevent complement-mediated hemolysis, we combined the identification of C5 epitopes using previously reported data and computational prediction with a modular strategy of VLP functionalization to produce candidate immunogens. These vaccine candidates were able to elicit anti-C5 autoantibodies and protect mice in a model of intravascular hemolysis. Our data indictes that autologous C5 vaccines could be developed as an alternative or supplement to Eculizumab for treating complement-mediated diseases such as PNH.

### Materials and Methods

### Mice and Complement Reagents

C57BL/6 WT mice were purchased from the Jackson Laboratory and maintained in the animal facility of Cleveland Clinic. All animal care and experimental procedures were approved by the Institutional Animal Care and Use Committee of Cleveland Clinic. Purified human C5 protein and pooled human C5-depleted serum were purchased from Complement Technology Inc (Tylor, Tex.)

### Mouse C5 Vaccine Design

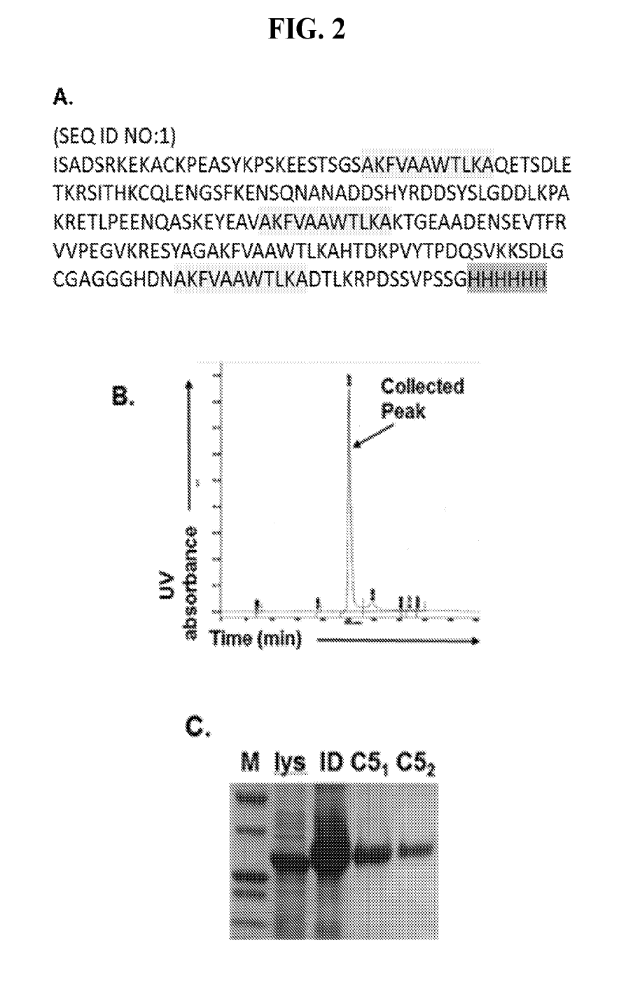

The mouse C5 protein sequence was analyzed using OptimumAntigen Design™ software (Genscript, NJ) which utilizes an advanced antigen design algorithm based on several protein databases. The identified epitopes were compared with the published C5 protein crystal Structure® to check for surface exposure. These steps provided twelve potential immunogenic epitopes that are likely present on the surface of the native mouse protein. An artificial gene was designed coding for a polypeptide comprised of these twelve potential epitopes interspersed in three places with a copy of the non-natural pan-DR epitope (PADRE) sequence (a linear peptide that improves humoral responses against antigens and helps break immune tolerance^{20,21}), and capped at the C-terminus with a 6×His tag. This is shown as SEQ ID NO:1 in FIG. 2.

### Mouse C5 Vaccine Expression and Purification

The custom-synthesized artificial gene (Genscript, NJ) was cloned into the PET-21b expression vector and transformed into E. coli strain BL21. Expression of the desired protein, intended to be a recombinant mouse C5 vaccine, was induced using the Overnight Express™ Autoinduction System (EMD Millipore, MA) following manufacturer provided protocols. The protein formed inclusion bodies and was isolated using the B-PER® Bacterial Protein Extraction Reagent (Thermo Fisher, IL), denatured in 8 M urea, and refolded by dialysis against PBS containing gradually reduced concentrations of urea. The refolded recombinant C5 was then affinity-purified using HisPur™ Cobalt Resin (Thermal Fisher, IL) following our previously published and manufacturer-provided protocols. The purity of the resultant protein was checked by SDS-PAGE and then purified again using a C8 reversed-phase HPLC (Beckman, Calif.). The single major protein peak was collected for sequential experiments.

### Mouse Immunizations

C57BL/6 WT mice (8-12 weeks old) were used in all in vivo experiments. The amounts administered per mouse were as follows: human C5, 25 μg; recombinant mouse C5, 100 μg; VLP-based C5, 200 μg. For immunization, vaccines were emulsified with complete Freund's adjuvant (CFA) (Difco Laboratories, MI), then administered subcutaneously. For human C5 and recombinant mouse C5 vaccine studies, mice immunized with CFA alone were included as controls; for VLP-based C5 vaccine studies, mice immunized with the same amount of wild-type (lacking the C5 epitope) VLPs were used as controls. The immunized mice were boosted 2 weeks after the initial immunization with the same amount of antigens in incomplete Freund's adjuvant (IFA) (Difco Laboratories, MI) once or twice after every other week.

### Serum Anti-C5 Antibody Titer Measurements by ELISA

One week after the last boost, serum was collected from the tail vein and the anti-C5 IgG titers were measured by ELISA. Briefly, the ELISA plate was coated with 5 μg/mL purified full-length human C5 or recombinant mouse C5. After blocking, diluted serum samples were added to the plate and incubated for 2 h. After washing, HRP-anti-mouse IgG was added as detective antibody. The absorbance was read at 450 nm using a microtiter plate reader (Molecular Devices, CA).

### Ex Vivo Mouse Complement-Mediated Hemolysis Assay

Antibody-sensitized sheep erythrocytes (E^{shA}) were used in the ex vivo mouse complement-mediated hemolysis assay following previously published protocol with modifications²². In brief, 15 μL mouse serum and 5 μL human C5 depleted serum were added to GVB⁺⁺ (5 mM Barbital, 145 mM NaCl, 0.5 mM MgCl₂, 0.15 mM CaCl₂ and 0.1% Gelatin, pH 7.4) in a total volume of 90 μL, then 10 μL of E^{shA} 5×10⁶) suspended in GVB⁺⁺ were added to each sample and incubated in 37° C. for 5 mins. For negative control, 5 mM EDTA was added to the GVB⁺⁺ to inhibit the complement activation. After incubation, samples were centrifuged, and the absorbance of the supernatant was measured at 414 nm using a microtiter plate reader. To calculate the percentage of hemolysis rate, the following equation was used: Hemolysis rate (%)=[(A−B)/(C−B)]×100%. A=OD₄₁₄ reading of sample in GVB⁺⁺, B=OD₄₁₄ reading of sample in GVB⁺⁺ with 5 mM EDTA, C=OD₄₁₄ reading of maximum hemolysis induced by H₂O.

### In Vivo Complement-Mediated Intravascular Hemolysis and Hemoglobinuria

Complement-induced intravascular hemolysis was induced followed by published protocols with modifications²³. In brief, each mouse was injected with 200 μL human C5-depleted serum intraperitoneally, and 1 hour later, blood was collected through retro-orbital bleeding (to minimize bleeding-related hemolysis). Sera were diluted at a 1:10 ratio in PBS in a total volume of 100 μL and hemolysis was quantitated by OD reading at 414 nm. At the same time of blood collection, the mouse abdomen was gently pressed to collect urine, which was diluted at a ratio of 1:10 in PBS before hemoglobin levels were measured by OD reading at 414 nm.

### Epitope Mapping of the Recombinant Mouse C5 Vaccine

A library of 15-mer oligopeptides overlapping each other by 6 amino acids, spanning the entire sequence of the recombinant mouse C5 vaccine, was custom-synthesized (GenScript, NJ) and used to map the dominant epitope(s) by ELISA. In brief, each peptide (20 μg/mL) was used to coat a plate at 4° C. overnight. After blocking with 1% BSA in PBS for two hours, the plate was incubated at room temperature for 2 h with 1:500 diluted sera collected from the recombinant C5 vaccine-immunized mice. After washing, the plate was incubated with HRP-labeled anti-mouse IgG at RT for 1 h, followed by color development by adding the HRP substrate TMB and OD₄₅₀ reading using a microplate reader (Molecular Devices).

### Mouse C5 Protein Structure Modeling and Peptides Epitope Localization

A structural homology model of mouse C5 was generated using the Phyre2 protein recognition server following established protocol³¹. The X-ray crystal structure of human C5 protein was used as the main template (PDB access code: 3cu7)¹⁹. Sequence homology between human and mouse C5 proteins is 89%. Normal modeling mode was used. Major steps of modeling include gathering homologous sequences, fold library scanning, loop modeling, and side-chain placement. The PyMOL visualization program (Schrödinger, LLC, MA) was used to display all the structural models in this work.

### Construction of Plasmids for VLP-C5 Vaccine

The previously described pCDF-CP plasmid²⁴ has a multiple cloning site (containing NdeI and XhoI restriction sequences) immediately downstream of the viral coat protein (CP) gene. The DNA sequence for the PADRE peptide, codon optimized for E. coli expression, was inserted at this site to generate plasmid pCDF-CP-PADRE. This was further elaborated with the codon-optimized DNA for the identified C5 epitope (ASYKPSKEESTSGS, SEQ ID NO:4, designated P2) downstream of the PADRE sequence, resulting in the plasmid pCDF-CP-PADRE-05.

### VLP-C5 Vaccine Production and Characterization

The VLP-C5 vaccine was produced and characterized following previously published protocols²⁴. In brief, electrocompetent ClearColi BL21(DE3) E. coli cells (Lucigen) were co-transformed with both pCDF-CP-PADRE-05 and pET28-CP to produce VLP-C5 particles. The control VLP particles were produced using only cells transformed with pET28-CP. Cells were plated on selective SOB agar. After 24 h, isolated colonies were selected into autoclaved SOB media (1% NaCl; 25 mL) containing appropriate antibiotics and grown overnight at 37° C. Cultures were diluted into autoclaved selective SOB media (1% NaCl; 500 mL) the following day. Cultures were grown at 37° C. until the cultures reached mid-log phase (O.D.₆₀₀˜0.9), at which time protein expression was induced by the addition of IPTG to a final concentration of 1 mM. Cultures were maintained at 30° C. for 16 h, after which cells were pelleted by centrifugation at 6,000 rpm for 10 min. Cells were lysed with a probe sonicator (10 min total, 5 s intervals) in an ice bath. Cell debris was removed by centrifugation at 14,000 rpm for 10 min, and supernatant collected. VLPs were precipitated by the addition of 30% (NH₄)₂SO₄ (w/v) at 4° C. for 2 h, and samples were pelleted at 14,000 rpm for 10 min. The pellet was dissolved in 1×TBS and extracted with n-BuOH:CHCl₃ (1:1, v/v) to remove lipids and aggregates. The samples were centrifuged at 3,500 rpm for 10 min, and the aqueous phase was collected and subsequently loaded onto 10-40% sucrose density gradients. VLPs purified on gradients by centrifugation at 28,000 rpm for 4 h, and VLP bands were isolated via syringe. Particles pelleted by centrifugation at 68,000 rpm for 2 h, and subsequent pellets dissolved in 0.1M phosphate buffer and characterized.

The resulting VLPs were characterized by FPLC size-exclusion chromatography (Superose 6, monitored by absorbance at 280 nm), dynamic light scattering (Wyatt Dynapro plate reader), and microfluidic gel electrophoresis (Agilent 2100 Bioanalyzer with Series II Protein 80 chips). The average number of CP and CP-05 subunits per particle was determined by integrating the electropherogram peaks in the instrument software. Levels of endotoxin contamination were found to be less than 0.1 EU/mL using Pierce LAL Chromogenic Endotoxin Quantitation Kit (Thermo Fisher).

### Results

Immunization with Human C5 Protein Raises Antibodies that do not Reduce Mouse C5 Activity

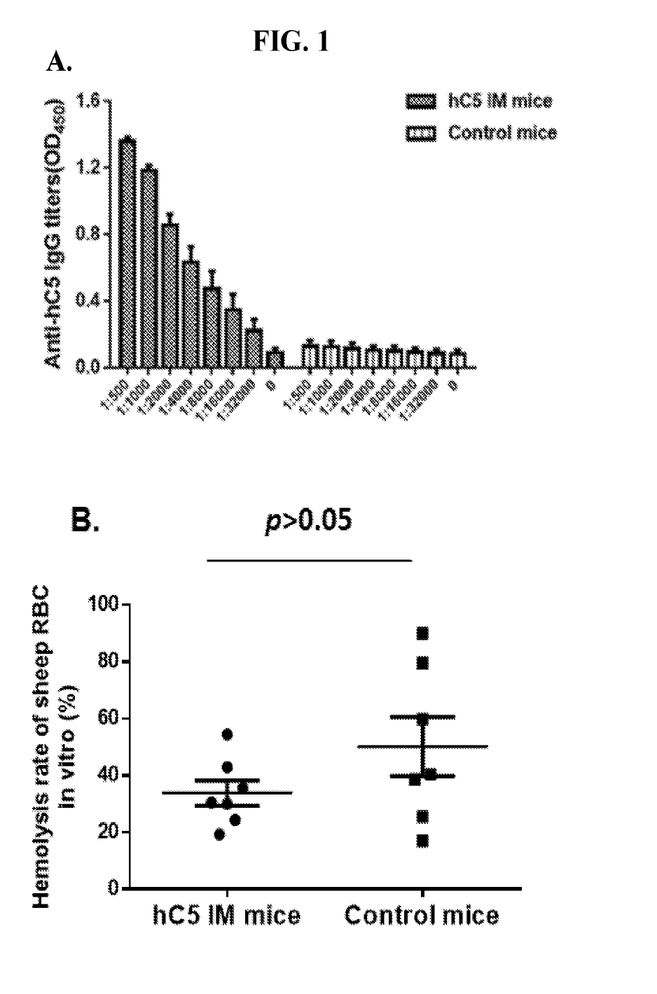

Because 1) autoantigens are generally immune tolerized, 2) previous studies suggest that conservative antigens from different species could elicit the production of antibodies that cross-react with the autoantigens²⁵⁻²⁹, and 3) mouse and human C5 protein share >80% homology³⁰, we initially immunized mice with purified human C5 protein, hoping that the developed polyclonal antibodies could cross-react with mouse C5, therefore inhibiting mouse C5 activity in vivo. Indeed, mice immunized with human C5 protein developed high titers of antibodies against human C5 (FIG. 1A), however, sera from these immunized mice had similar potency as sera from control mice in lysing sensitized sheep erythrocytes ex vivo (FIG. 1B). These results demonstrated that immunizing mice with human C5 did not develop cross-reacting antibodies to reduce mouse C5 activity in vivo.

Design and Production of an Autologous C5 Vaccine with Predicted Protein Surface Epitopes Together with the PADRE Peptide

We then planned to immunize the mice with an autologous mouse C5 vaccine to develop mouse anti-mouse C5 autoantibodies with the goal of reducing or blocking C5 activity as in patients receiving Eculizumab. Analysis of the mouse C5 protein sequences identified 12 potential immunogenic epitopes that are likely to be on the surface of the protein (important so that the generated antibodies can have access to them under native conditions) (Table 1).

TABLE 1

| Peptide | | Antigenic | SEQ ID | | Antigenicity/Surface/ |
|---|---|---|---|---|---|
| No. | Start | Determinant | NO: | Length | Hydrophilicity |
| P1 | 1547 | ISADSRKEKACKPE | 3 | 14 | 2.66/0.79/1.14 |
| P2 | 1410 | ASYKPSKEESTSGS | 4 | 14 | 2.56/0.86/0.69 |
| P3 | 399 | QETSDLETKRSITH | 5 | 14 | 2.42/0.86/0.85 |
| P4 | 1114 | KCQLENGSFKENSQ | 6 | 14 | 2.38/0.79/0.43 |
| P5 | 657 | NANADDSHYRDDS | 7 | 14 | 2.32/0.79/0.86 |
| P6 | 146 | YSLGDDLKPAKRET | 8 | 14 | 2.29/0.71/0.73 |
| P7 | 442 | LPEENQASKEYEAV | 9 | 14 | 2.22/0.71/0.76 |
| P8 | 1590 | KTGEAADENSEVTF | 10 | 14 | 2.19/0.86/0.65 |
| P9 | 932 | RVVPEGVKRESYAG | 11 | 14 | 2.17/0.71/0.43 |
| P10 | 630 | KSDLGCGAGGGHDN | 12 | 14 | 1.86/0.93/0.38 |
| P11 | 129 | HTDKPVYTPDQSVK | 13 | 14 | 1.80/0.86/0.22 |
| P12 | 1234 | DTLKRPDSSVPSSG | 14 | 14 | 1.73/0.86/0.58 |

We designed an artificial gene (SEQ ID NO:1) coding for all of these identified epitopes in a linear sequence together with three copies of a previously published tolerance-breaking peptide, PADRE^{20,21} (FIG. 2A). This polypeptide was expressed in E. coli, isolated from inclusion bodies, denatured, refolded, affinity purified via a 6×His tag (FIG. 2B), and finally purified by reversed-phase HPLC (FIG. 2C).
Mice Immunized with the Autologous C5 Vaccine Develop C5-Reactive Autoantibodies and Show Decreased Complement Hemolytic Activity

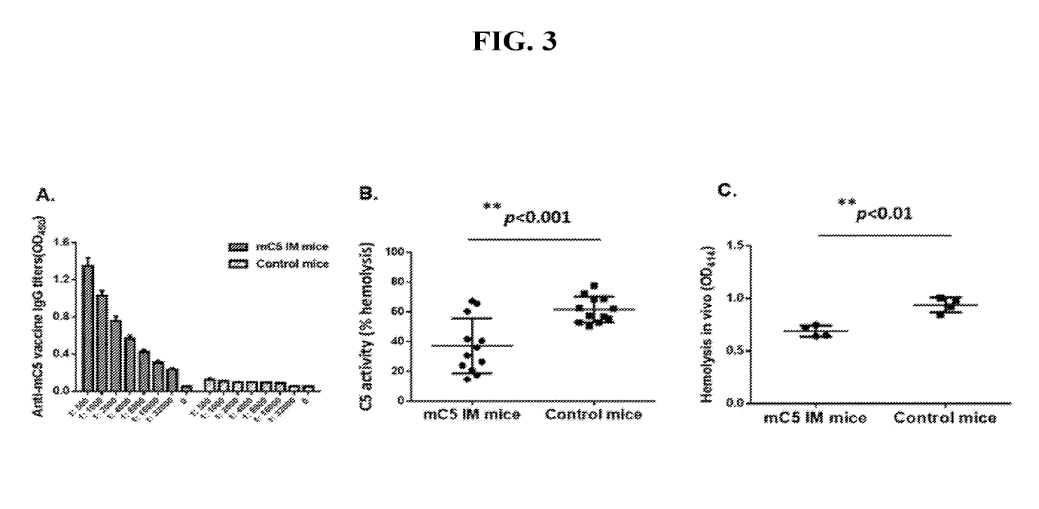

Mice that were immunized and boosted with the purified C5 multi-epitope polypeptide showed high IgG antibody titers against full-length recombinant mouse C5 protein by ELISA (FIG. 3A). Therefore, this C5 vaccine was able to break immune tolerance and develop a significant immune response to the autologous C5 epitopes. Furthermore, the mice receiving the C5-based polypeptide vaccine showed less complement-mediated hemolytic activity than the control mice (FIG. 3B, 3C). This suggests that the recombinant autologous C5 vaccine is effective in eliciting functional anti-C5 autoantibodies that have the intended outcome of inhibiting C5 activity.

Identification of the Dominant Epitopes within the Autologous C5 Vaccine Using a Synthesized Overlapping Peptide Library

To identify which of the 12 C5 peptide sequences were the most effective in inducing the above immune response, we designed a library of overlapping 15-mer oligopeptides spanning the entire sequence of the recombinant C5 vaccine (Table 2).

TABLE 2

| SEQ ID NO: | Peptide |
|---|---|
| 15 | ISADSRKEKACKPEA |
| 16 | ACKPEASYKPSKEES |
| 17 | PSKEESTSGSAKFVA |
| 18 | SAKFVAAWTLKAQET |
| 19 | LKAQETSDLETKRSI |
| 20 | ETKRSITHKCQLENG |
| 21 | CQLENGSFKENSQNA |
| 22 | ENSQNANADDSHYRD |
| 23 | DSHYRDDSYSLGDDL |
| 24 | SLGDDLKPAKRETLP |
| 25 | KRETLPEENQASKEY |
| 26 | QASKEYEAVAKFVAA |
| 27 | AKFVAAWTLKAKTGE |
| 28 | KAKTGEAADENSEVT |
| 29 | ENSEVTFRVVPEGVK |
| 30 | VPEGVKRESYAGAKF |
| 31 | YAGAKFVAAWTLKAH |
| 32 | WTLKAHTDKPVYTPD |
| 33 | PVYTPDQSVKKSDLG |
| 34 | KKSDLGCGAGGGHDN |
| 35 | GGGHDNAKFVAAVVTL |
| 36 | VAAWTLKADTLKRPD |
| 37 | TLKRPDSSVPSSG |
| 38 | DTLKRPDSSVPSSG |

Using these individual peptides as ELISA probes, we assessed the IgG composition of the serum from the C5 vaccine-immunized mice and found that the sera contained high titers of IgGs against the peptide ACKPEASYKPSKEES (SEQ ID NO:39), suggesting that this sequence contains a dominant surface epitope within the vaccine that elicited strong humoral responses.

### Visualizing the C5 Peptide Containing the Identified Dominant Epitope on Mouse C5 Three-Dimensional Structure Model

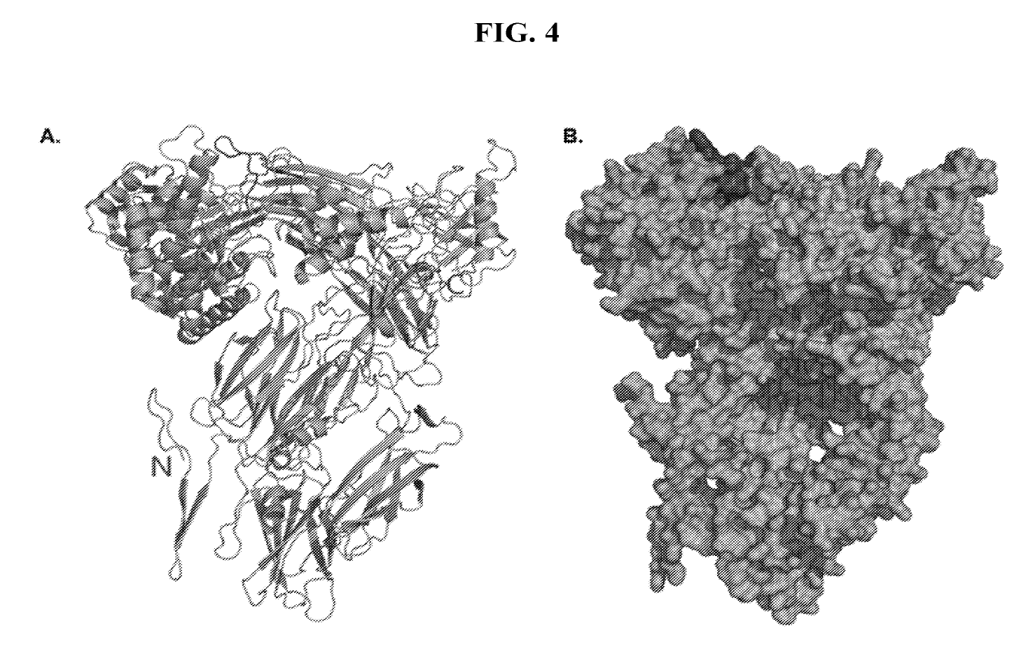

The C5 peptide P2 (ASYKPSKEESTSGS (SEQ ID NO: 4)) is originally predicted by the computer algorithms to be an immunogenic epitope exposed on the C5 protein surface and it contains the peptide sequence identified in the above-described epitope mapping experiments. To verify that this peptide is indeed on C5 protein surface for our future VLP-05 vaccine development, we constructed a mouse C5 three-dimensional homology model based on the published human C5 crystal Structure® using the Phyre2 protein recognition program following published protocols³¹. Mouse C5 protein shares 89% homology with the human C5 sequence, providing a model with high confidence (100%) and high sequence coverage (98%). Visualization of the location of the P2 peptide in the mouse C5 structure model showed it to be exposed on the solvent-exposed surface (FIG. 4, shaded dark), further supporting the hypothesis that it represents a suitable epitope for the development of VLP-C5 vaccines.

### Preparation of a VLP-Based C5 Vaccine Displaying the C5 Epitope (P2)

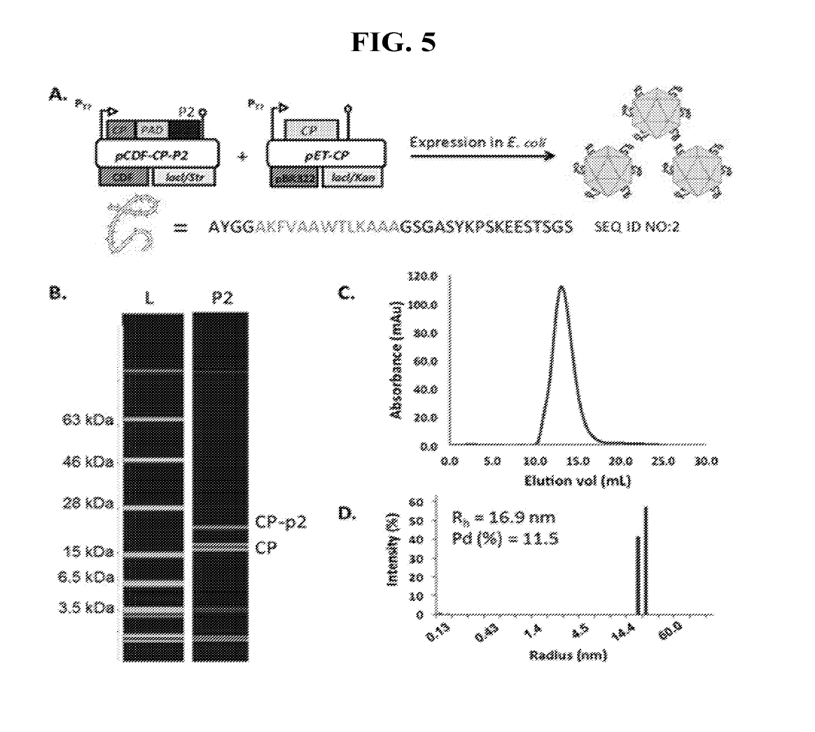

A recombinant VLP bearing a linear peptide composed of the putative dominant C5 surface epitope P2 and the PADRE peptide (separated by a short spacer) was expressed as a C-terminal extension of the VLP coat protein (FIG. 5A). These particles were produced by a well-established method²⁴ in which a mixture of truncated and extended capsid proteins are co-expressed in E. coli and self-assemble into a hybrid nanoparticle in which an average of 50 copies of the extension were incorporated per capsid. The resultant VLP-C5 vaccine was characterized using dynamic light scattering, size-exclusion chromatography, gel electrophoresis, and high-resolution mass spectrometry (FIGS. 5B, C&D), showing intact particles of the expected size (˜30 nanometers) and composition.

Mice Immunized with the VLP-C5 Vaccine Developed Antibodies Against the Recombinant Mouse C5 and Showed Reduced Hemolytic Activity

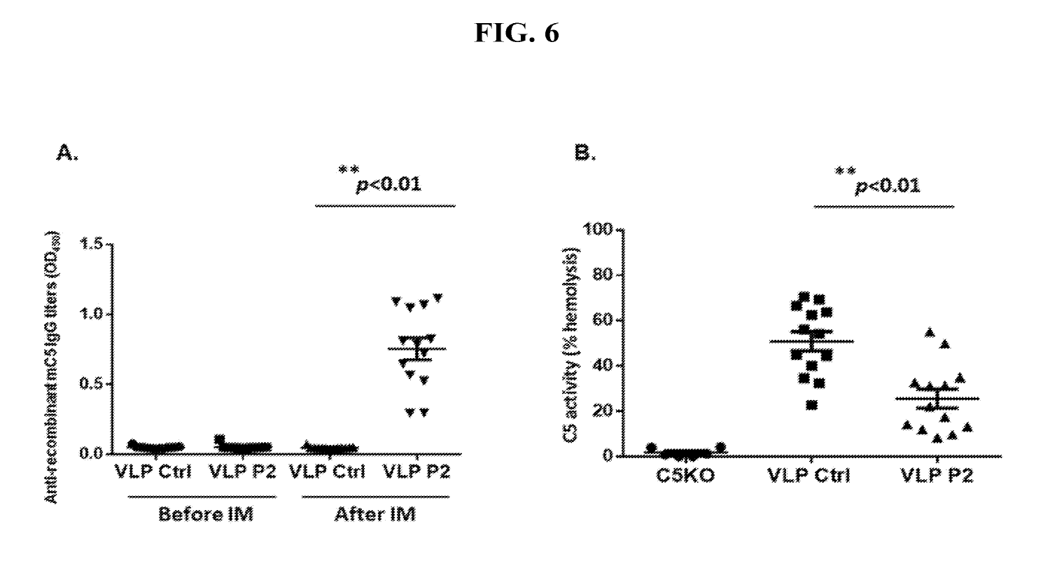

WT mice were immunized with either the VLP-C5 vaccine or the same amount of control VLP lacking the extended capsid component. Using the same analyses as described above, we found that mice receiving the control VLP produced no detectable recombinant mouse C5-reactive IgGs (FIG. 6A), whereas the VLP-C5 vaccine immunized mice developed high titers of IgG antibodies against intact recombinant mouse C5. Most importantly, the complement-mediated hemolytic activity of these VLP-C5 vaccine-immunized mice was also significantly reduced (FIG. 6B), indicating that this VLP-based vaccine is effective in eliciting C5-reactive autoantibody production and reducing C5 activity.

Mice Immunized with the VLP Vaccine are Protected in a Model of Complement-Mediated Hemolytic Anemia

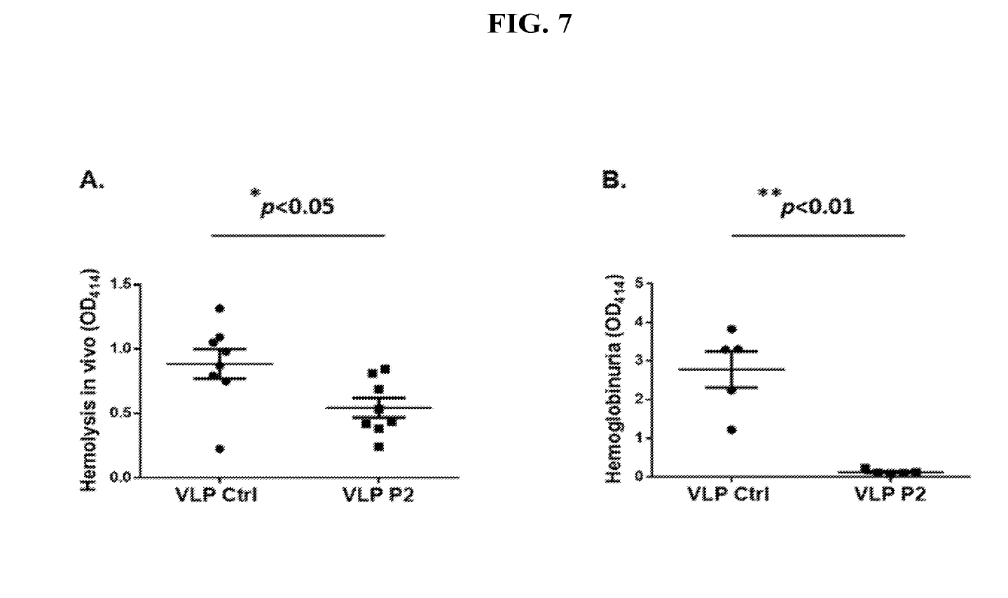

To test whether the C5 vaccine approach is effective in protecting mice from complement-mediated intravascular hemolysis, as found in patients with PNH, we modified a previously established complement-mediated intravascular hemolytic anemia model²³. In brief, we introduced 200 μL of C5-depleted human serum into each of the VLP-C5 vaccine-immunized mice and control mice by i.p. injection. After one hour, each mouse was bled retro-orbitally to collect sera to measure intravascular hemolysis, and urine was also collected to measure hemoglobinuria. We found that injection of C5-depleted human serum-induced much less severe hemolysis and hemoglobinuria in the VLP-C5 vaccine-immunized mice compared to the control mice (FIG. 7). This suggests that mouse C5 can replace human C5 to form functional MAC and that the VLP-C5 vaccine is effective in protecting mice from complement-mediated cell damage in vivo.

Results with P3 and P12

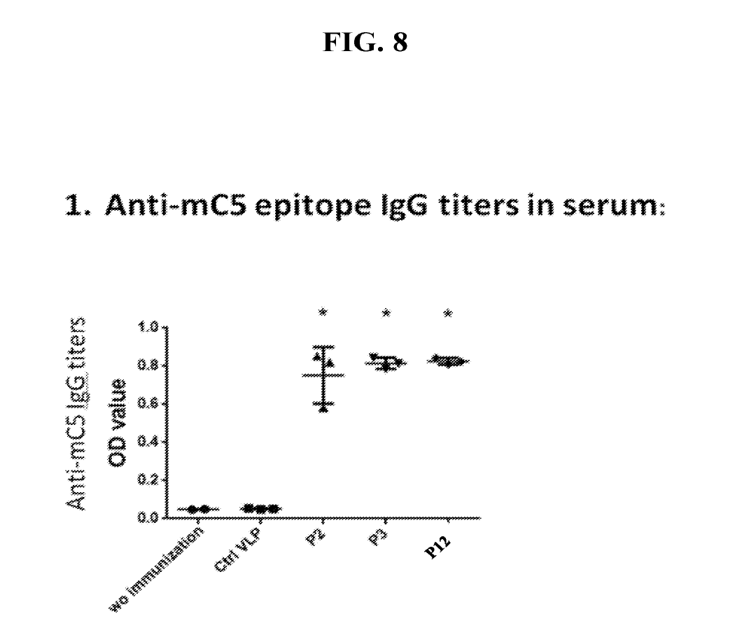

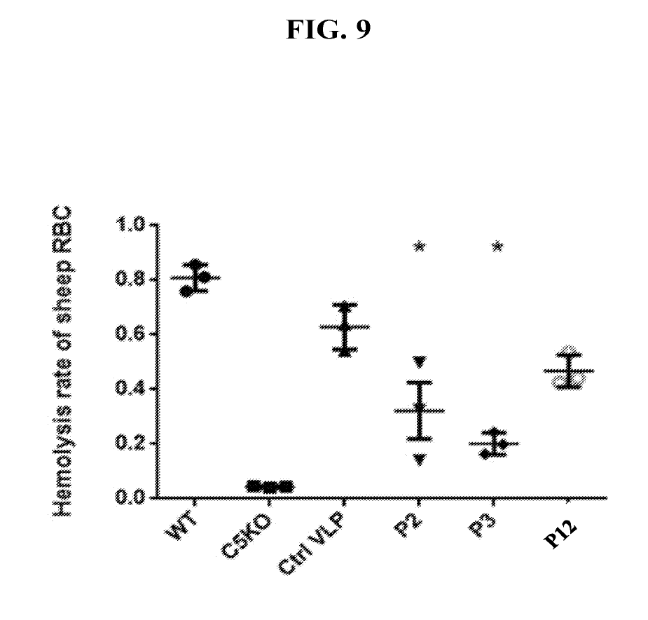

The P3 and P12 peptides were tested in the same manner as the P2 peptide as described above. The results of this testing are shown in FIGS. 8 and 9. Figures shows that VLPs displaying P2, P3 or P12 elicit mouse anti mouse C5 antibodies. Sera collected from mice without immunization (wo immunization), mice immunized with VLPs displaying different C5 epitopes (P2, P3 or P12) or the control empty VLPs (Ctrl VLP) were assessed for mouse C5-reactive IgG levels by conventional ELISA. FIG. 9 shows that immunization of VLPs displaying P2 or P3 reduces blood C5 activities ex vivo. Sera collected from C5 deficient mice (C5KO), mice without immunization (WT), mice immunized with VLPs displaying different C5 epitopes (P2, P3 or P12) or the control empty VLPs (Ctrl VLP) were assessed for C5 activity using a modified sheep RBC-based C5 activity assay.

Aberrant activity of the complement system is emerging as a target for treating certain inflammatory and autoimmune diseases³². While excess complement activity can have many potential causes, a choke point of the system is the C5 protein, responsible for the penultimate step in the induction of hemolytic function. Most of the complement-targeted reagents that have been approved or under development are mAbs³³; others are based on aptamers³⁴, peptides^{35,36}, and small molecules³⁷. Given the life-long complement inhibition required in most of these patients, all currently available complement inhibition therapies suffer from limited half-life, high costs, and compliance issues. This is especially true in the case of the anti-C5 antibody ECULIZUMAB, which many PNH patients have to receive by i.v. infusion every two weeks for life, at a current price of almost a half million dollars a year per patient^{3,38}. Interest in C5 as a target has recently expanded to other diseases, with ECULIZUMAB in late-stage clinical trials for conditions such as myasthenia gravis³⁹ and cold agglutinin disease⁴⁰ that involve excessive complement activation.

We show here examples of C5 vaccine-based approaches for the production of autologous C5-reactive antibodies to reduce C5 activity for the treatment of complement-mediated diseases (e.g., mimicking the effect of ECULIZUMAB). Vaccination with a homologous C5 from a different species (human) did not confer anti-mouse C5 function in mouse, although it did elicit a strong antibody response against the immunized human C5. This stands in contrast to many examples of the use of conservative xeno-antigens in antitumor vaccines, in which immunization elicited T- and/or B cell responses that cross-react with their mouse counterparts on tumor cells²⁶⁻²⁹. In our case, we suggest that the overlapping epitopes between mouse and human C5 (which are more than 80% homologous) are well-tolerated in mice and that all the antibodies produced were likely against the epitopes that are unique in the human C5.

Instead, we were able to break tolerance in mouse using a linear polypeptide composed of all the computer-predicted strong immunogenic epitopes along with the PADRE sequence. We identified a potential dominant C5 peptide within these 12 computer-predicted immunogenic peptides by epitope mapping using an overlapping peptide library and constructed a mouse C5 structure model to ensure that this identified C5 peptide is exposed on the mouse C5 protein surface. We then developed VLP nanoparticles that display this dominant C5 surface peptide together with the PADRE sequence as a new autologous C5 vaccine. The VLP-based C5 vaccine proved to be especially effective at eliciting autologous anti-C5 antibody production, inhibiting C5 activity, and protecting the vaccinated mice from intravascular hemolysis and hemoglobinuria in a model that mimics PNH. Our results suggest that autologous C5 vaccines may be a supplement and/or alternative for ECULIZUMAB for treating complement-mediated diseases.

Vaccination against C5 to have the immune system produce its own anti-C5 antibodies represents a cost- and compliance-effective alternative to antibody infusion, with therapeutic effects prolonged by booster immunizations. Indeed, one publication has appeared testing the potential of a vaccination approach against complement in treating/preventing diseases⁴¹. In that work, a peptide of undisclosed sequence was designed to mimic one or more mouse C5a epitopes, and immunization of this peptide conjugated to carrier protein KLH elicited anti-C5a autoantibodies and protected the vaccinated mice from chronic neuroinflammation and neuropathologic alterations in a model of Alzheimer's disease⁴¹. This work supports the concept of complement-targeted vaccination for treating complement-mediated diseases, but it also significantly differs from the work presented here. The vaccine tested in the prior report was specific to C5a, which is a complement activation product released from activated C5, and therefore does not have an effect on C5b-9 (MAC) formation. In contrast, the C5 vaccines developed here do not target C5a, but are specific for the C5b part of C5, which is the essential component to initiate the assembly of the MAC to lyse RBCs and to damage other tissues in pathological conditions.

Another important difference in these approaches is the relative concentrations of the target. The C5a protein exists in blood at very low levels (˜0.5-50 ng/mL)^{42,43,} whereas C5 concentration is approximately 100-200 μg/mL in the blood. It requires strong vaccines to break the tolerance to produce enough anti-C5 antibodies to significantly reduce C5 activity in vivo. By combining multiple epitopes and the PADRE peptide, and by using a VLP as a carrier, our approaches did indeed develop high titers of anti-C5 autoantibodies and decreased C5 activity. However, C5 activity was not completely eliminated in the immunized mice. This is significant because of the dangers inherent in the complete elimination of complement function. An eventual therapeutic application of this vaccine strategy would require a moderation, but not elimination, of C5 activity, which seems to be possible on the basis of this initial study.

Our use of the PADRE peptide was stimulated by its known ability to help break immune tolerance and to improve humoral responses against immunized antigens both in humans and in C57BL/6 mice^{20,21}. PADRE works by strongly binding to 15 of 16 of the most common HLA-DR types tested to date in humans and I-A^{b} molecules in mice to boost T help responses to improve immune responses against both T cell- and B cell epitopes. Tests to support or refute the assumption that these mechanistic factors are important in the present application must await further studies.

It should also be noted that the use of human C5-depleted serum to induce intravascular hemolysis is a new model of situations found in PNH patients. Mouse complement is much weaker than human complement in hemolytic activity. The severe intravascular hemolysis and hemoglobinuria induced by injecting mice with C5-depleted serum, which does not have any hemolytic activity by itself, shows that mouse C5 can replace human C5 to form “hybrid” MAC to lyse mouse RBCs in vivo. We believe this model will be useful in other proof-of-concept studies testing mouse C5 targeted therapies in treating complement-mediated hemolysis or tissue damage.

As detailed above, it was found that immunization of the conservative human C5 did not raise cross-reactive antibodies that reduced mouse C5 activity. We showed that immunization of a recombinant mouse C5 vaccine composed of multiple potential protein surface B cell epitopes and the PADRE peptides broke immune tolerance of C5 in mice and decreased C5 activity. Finally, we demonstrated that immunization of a VLP vaccine displaying one of the identified strong mouse C5 surface epitopes together with the PADRE peptide elicited strong humoral responses against mouse C5, reduced C5 activity and protected mice from complement-mediated hemolysis and hemoglobinuria in a model of intravascular hemolysis, which closely mimic the situations found in PNH patients. Our results suggest that C5 auto-vaccines, especially the ones on the VLP platform, could supplement and/or be an alternative for ECULIZUMAB for treating complement-mediated diseases.

### REFERENCES

1. Ricklin D, Hajishengallis G, Yang K, Lambris J D. Complement: a key system for immune surveillance and homeostasis. Nat Immunol. 2010; 11(9):785-797.
2. Ricklin D, Lambris J D. Progress and trends in complement therapeutics. Adv Exp Med Biol. 2013; 735:1-22.
3. Hillmen P, Hall C, Marsh J C, et al. Effect of eculizumab on hemolysis and transfusion requirements in patients with paroxysmal nocturnal hemoglobinuria. N Engl J Med. 2004; 350(6):552-559.
4. Zuber J, Fakhouri F, Roumenina L T, Loirat C, Fremeaux-Bacchi V, French Study Group for a HCG. Use of eculizumab for atypical haemolytic uraemic syndrome and C3 glomerulopathies. Nat Rev Nephrol. 2012; 8(11):643-657.
5. DeZern A E, Brodsky R A. Paroxysmal Nocturnal Hemoglobinuria: A Complement-Mediated Hemolytic Anemia. Hematol Oncol Clin North Am. 2015; 29(3):479-494.
6. Coyle D, Cheung M C, Evans G A. Opportunity cost of funding drugs for rare diseases: the cost-effectiveness of eculizumab in paroxysmal nocturnal hemoglobinuria. Med Decis Making. 2014; 34(8):1016-1029.
7. Murphy R M. Rare diseases mean big profits. Fortune. 2012; 166(4):24.
8. Bachmann M F, Jennings G T. Vaccine delivery: a matter of size, geometry, kinetics and molecular patterns. Nat Rev Immunol. 2010; 10(11):787-796.
9. Chackerian B. Virus-like particle based vaccines for Alzheimer disease. Human Vaccines. 2010; 6(11):926-930.
10. Handisurya A, Gilch S, Winter D, et al. Vaccination with prion peptide-displaying papillomavirus-like particles induces autoantibodies to normal prion protein that interfere with pathologic prion protein production in infected cells. FEBS J. 2007; 274(7):1747-1758.
11. Spohn G, Schwarz K, Maurer P, et al. Protection against osteoporosis by active immunization with TRANCE/RANKL displayed on virus-like particles. J Immunol. 2005; 175(9):6211-6218.
12. Li Q, Cao C, Chackerian B, et al. Overcoming antigen masking of anti-amyloidbeta antibodies reveals breaking of B cell tolerance by virus-like particles in amyloidbeta immunized amyloid precursor protein transgenic mice. BMC Neurosci. 2004; 5:21.
13. Bachmann M F, Jennings G T. Therapeutic vaccines for chronic diseases: successes and technical challenges. Philos Trans R Soc Lond B Biol Sci. 2011; 366(1579):2815-2822.
14. Tissot A C, Maurer P, Nussberger J, et al. Effect of immunisation against angiotensin II with CYT006-AngQb on ambulatory blood pressure: a double-blind, randomised, placebo-controlled phase IIa study. Lancet. 2008; 371(9615):821-827.
15. Senti G, Johansen P, Haug S, et al. Use of A-type CpG oligodeoxynucleotides as an adjuvant in allergen-specific immunotherapy in humans: a phase I/IIa clinical trial. Clin Exper Allergy. 2009; 39(4):562-570.
16. Ambuhl P M, Tissot A C, Fulurija A, et al. A vaccine for hypertension based on virus-like particles: preclinical efficacy and phase I safety and immunogenicity. J Hypertens. 2007; 25(1):63-72.
17. Tissot A C, Spohn G, Jennings G T, et al. A VLP-based vaccine against interleukin-1alpha protects mice from atherosclerosis. Eur J Immunol. 2013; 43(3):716-722.
18. Brown S D, Fiedler J D, Finn M G. Assembly of hybrid bacteriophage Qbeta virus-like particles. Biochemistry. 2009; 48(47):11155-11157.
19. Fredslund F, Laursen N S, Roversi P, et al. Structure of and influence of a tick complement inhibitor on human complement component 5. Nat Immunol. 2008; 9(7):753-760.
20. Alexander J, del Guercio M F, Maewal A, et al. Linear PADRE T helper epitope and carbohydrate B cell epitope conjugates induce specific high titer IgG antibody responses. J Immunol. 2000; 164(3):1625-1633.
21. del Guercio M F, Alexander J, Kubo R T, et al. Potent immunogenic short linear peptide constructs composed of B cell epitopes and Pan DR T helper epitopes (PADRE) for antibody responses in vivo. Vaccine. 1997; 15(4):441-448.
22. Tu Z, Bu H, Dennis J E, Lin F. Efficient osteoclast differentiation requires local complement activation. Blood. 2010; 116(22):4456-4463.
23. Ino Y, Sato T, Suzuki S, Iwaki M, Yoshikawa T. Inhibitory effects of FUT-175, a new synthetic protease inhibitor, on intravascular hemolysis by human serum in mice. Int J Immunopharmacol. 1987; 9(5):533-537.
24. Fiedler J D, Higginson C, Hovlid M L, et al. Engineered mutations change the structure and stability of a virus-like particle. Biomacromolecules. 2012; 13(8):2339-2348.
25. Fattori E, Cappelletti M, Lo Surdo P, et al. Immunization against proprotein convertase subtilisin-like/kexin type 9 lowers plasma LDL-cholesterol levels in mice. J Lipid Res. 2012; 53(8):1654-1661.
26. Fong L, Brockstedt D, Benike C, et al. Dendritic cell-based xenoantigen vaccination for prostate cancer immunotherapy. J Immunol. 2001; 167(12):7150-7156.
27. Su J M, Wei Y Q, Tian L, et al. Active immunogene therapy of cancer with vaccine on the basis of chicken homologous matrix metalloproteinase-2. Cancer Res. 2003; 63(3):600-607.
28. Wei Y Q, Huang M J, Yang L, et al. Immunogene therapy of tumors with vaccine based on Xenopus homologous vascular endothelial growth factor as a model antigen. Proc Natl Acad Sci USA. 2001; 98(20):11545-11550.
29. Wei Y Q, Wang Q R, Zhao X, et al. Immunotherapy of tumors with xenogeneic endothelial cells as a vaccine. Nat Med. 2000; 6(10):1160-1166.
30. Haviland D L, Haviland J C, Fleischer D T, Wetsel R A. Structure of the murine fifth complement component (C5) gene. A large, highly interrupted gene with a variant donor splice site and organizational homology with the third and fourth complement component genes. J Biol Chem. 1991; 266(18):11818-11825.
31. Kelley L A, Mezulis S, Yates C M, Wass M N, Sternberg M J. The Phyre2 web portal for protein modeling, prediction and analysis. Nat Protoc. 2015; 10(6):845-858.
32. Ricklin D, Lambris J D. New milestones ahead in complement-targeted therapy. Semin Immunol. 2016.
33. Ricklin D, Lambris J D. Complement in immune and inflammatory disorders: therapeutic interventions. J Immunol. 2013; 190(8):3839-3847.
34. Hoehlig K, Maasch C, Shushakova N, et al. A novel C5a-neutralizing mirror-image (l-)aptamer prevents organ failure and improves survival in experimental sepsis. Mol Ther. 2013; 21(12):2236-2246.
35. Sharp J A, Whitley P H, Cunnion K M, Krishna N K. Peptide inhibitor of complement c1, a novel suppressor of classical pathway activation: mechanistic studies and clinical potential. Front Immunol. 2014; 5:406.
36. Sahu A, Morikis D, Lambris J D. Compstatin, a peptide inhibitor of complement, exhibits species-specific binding to complement component C3. Mol Immunol. 2003; 39(10):557-566.
37. Ames R S, Lee D, Foley J J, et al. Identification of a selective nonpeptide antagonist of the anaphylatoxin C3a receptor that demonstrates antiinflammatory activity in animal models. J Immunol. 2001; 166(10):6341-6348.
38. Risitano A M, Marotta S. Therapeutic complement inhibition in complement-mediated hemolytic anemias: Past, present and future. Semin Immunol. 2016.
39. Howard J F, Jr., Barohn R J, Cutter G R, et al. A randomized, double-blind, placebo-controlled phase II study of eculizumab in patients with refractory generalized myasthenia gravis. Muscle Nerve. 2013; 48(1):76-84.
40. Shapiro R, Chin-Yee I, Lam S. Eculizumab as a bridge to immunosuppressive therapy in severe cold agglutinin disease of anti-Pr specificity. Clin Case Rep. 2015; 3(11):942-944.
41. Landlinger C, Oberleitner L, Gruber P, et al. Active immunization against complement factor C5a: a new therapeutic approach for Alzheimer's disease. J Neuroinflammation. 2015; 12:150.
42. Mocco J, Wilson D A, Komotar R J, et al. Alterations in plasma complement levels after human ischemic stroke. Neurosurgery. 2006; 59(1):28-33; discussion 28-33.
43. Gressner O A, Koch A, Sanson E, Trautwein C, Tacke F. High C5a levels are associated with increased mortality in sepsis patients—no enhancing effect by actin-free Gc-globulin. Clin Biochem. 2008; 41(12):974-980.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A composition comprising an immunogen, or a nucleic acid sequence encoding said immunogen, wherein said immunogen comprises at least one complement component 5 (C5) epitope.

2. The composition of claim 1, wherein C5 epitope is a C5b epitope.

3. The composition of claim 1, wherein said immunogen further comprises a PADRE sequence.

4. The composition of claim 1, wherein said immunogen further comprises a virus coat protein.

5. The composition of claim 1, wherein said C5 epitope is formed by an amino acid sequence comprising, consisting essentially of, or consisting of SEQ ID NOS:3-76, or an amino acid sequence with at least 90% sequence identity to SEQ ID NOS:3-76.

6. The composition of claim 5, wherein said C5 epitope is the only C5 derived sequence in said immunogen, and wherein said amino acid sequence is between 10 and 30 amino acids in length.

7. The composition of claim 5, wherein said C5 epitope is the only C5 derived sequence in said immunogen, and wherein said amino acid sequence is between 12 and 25 amino acids in length.

8. The composition of claim 1, wherein said immunogen further comprises a virus like particle.

9. The composition of claim 1, wherein said at least one C5 epitope is at least two C5 epitopes.

10. The composition of claim 1, wherein said at least one C5 epitope is a least 10 C5 epitopes.

11. The composition of claim 1, further comprising an immune-effective amount of an immunostimulant.

12. The composition of claim 11, wherein said immunostimulant comprises an adjuvant selected from the group consisting of: Freunds adjuvant, alum, aluminum hydroxide, aluminum phosphate, and calcium phosphate hydroxide.

13. The composition of claim 1, wherein said C5 epitope is formed by an amino acid sequence comprising, consisting essentially of, or consisting of SEQ ID NOS:40-76.

14. (canceled)

15. A composition comprising a nanoparticle or virus like particle, wherein said nanoparticle or said virus like particle comprises multiple copies of a first peptide arrayed thereon, wherein said first peptide comprises at least one complement component 5 (C5) epitope.

16-19. (canceled)

20. A method of immunizing a subject comprising:
administering a composition to a subject, wherein said composition comprises an immunogen, or a nucleic acid sequence encoding said immunogen, wherein said immunogen comprises at least one complement component 5 (C5) epitope, and wherein said administering causes autologous anti-C5 antibodies to be generated in said subject.

21. The method of claim 20, wherein said subject is affected with a human complement component 5 (C5) related disease.

22. The method of claim 20, wherein said immunogen further comprises a PADRE sequence.

23. The method of claim 20, wherein said immunogen further comprises a virus coat protein.

24. The method of claim 20, wherein said C5 epitope is formed by an amino acid sequence comprising, consisting essentially of, or consisting of SEQ ID NOS:3-76, or an amino acid sequence with at least 90% sequence identity to SEQ ID NOS:3-76.

25. The method of claim 20, wherein said C5 epitope is formed by an amino acid sequence comprising, consisting essentially of, or consisting of SEQ ID NOS:40-76.


SEQUENCE LISTING (76 sequences)

SEQ ID NO: 1 - protein, 217 aa, Artificial sequence
  Synthetic peptide
ISADSRKEKACKPEASYKPSKEESTSGSAKFVAAWTLKAQETSDLETKRSITHKCQLENGSFKENSQNANADDSHYRDDSYSLGDDLKPAKRETLPEENQASKEYEAVAKFVAAWTLKAKTGEAADENSEVTFRVVPEGVKRESYAGAKFVAAWTLKAHTDKPVYTPDQSVKKSDLGCGAGGGHDNAKFVAAWTLKADTLKRPDSSVPSSGHHHHHH

SEQ ID NO: 2 - protein, 34 aa, Artificial sequence
  Synthetic peptide
AYGGAKFVAAWTLKAAAGSGASYKPSKEESTSGS

SEQ ID NO: 3 - protein, 14 aa, Mus musculus
ISADSRKEKACKPE

SEQ ID NO: 4 - protein, 14 aa, Mus musculus
ASYKPSKEESTSGS

SEQ ID NO: 5 - protein, 14 aa, Mus musculus
QETSDLETKRSITH

SEQ ID NO: 6 - protein, 14 aa, Mus musculus
KCQLENGSFKENSQ

SEQ ID NO: 7 - protein, 13 aa, Mus musculus
NANADDSHYRDDS

SEQ ID NO: 8 - protein, 14 aa, Mus musculus
YSLGDDLKPAKRET

SEQ ID NO: 9 - protein, 14 aa, Mus musculus
LPEENQASKEYEAV

SEQ ID NO: 10 - protein, 14 aa, Mus musculus
KTGEAADENSEVTF

SEQ ID NO: 11 - protein, 14 aa, Mus musculus
RVVPEGVKRESYAG

SEQ ID NO: 12 - protein, 14 aa, Mus musculus
KSDLGCGAGGGHDN

SEQ ID NO: 13 - protein, 14 aa, Mus musculus
HTDKPVYTPDQSVK

SEQ ID NO: 14 - protein, 14 aa, Mus musculus
DTLKRPDSSVPSSG

SEQ ID NO: 15 - protein, 15 aa, Artificial sequence
  Synthetic peptide
ISADSRKEKACKPEA

SEQ ID NO: 16 - protein, 15 aa, Artificial sequence
  Synthetic peptide
ACKPEASYKPSKEES

SEQ ID NO: 17 - protein, 15 aa, Artificial sequence
  Synthetic peptide
PSKEESTSGSAKFVA

SEQ ID NO: 18 - protein, 15 aa, Artificial sequence
  Synthetic peptide
SAKFVAAWTLKAQET

SEQ ID NO: 19 - protein, 15 aa, Artificial sequence
  Synthetic peptide
LKAQETSDLETKRSI

SEQ ID NO: 20 - protein, 15 aa, Artificial sequence
  Synthetic peptide
ETKRSITHKCQLENG

SEQ ID NO: 21 - protein, 15 aa, Artificial sequence
  Synthetic peptide
CQLENGSFKENSQNA

SEQ ID NO: 22 - protein, 15 aa, Artificial sequence
  Synthetic peptide
ENSQNANADDSHYRD

SEQ ID NO: 23 - protein, 15 aa, Artificial sequence
  Synthetic peptide
DSHYRDDSYSLGDDL

SEQ ID NO: 24 - protein, 15 aa, Artificial sequence
  Synthetic peptide
SLGDDLKPAKRETLP

SEQ ID NO: 25 - protein, 15 aa, Artificial sequence
  Synthetic peptide
KRETLPEENQASKEY

SEQ ID NO: 26 - protein, 15 aa, Artificial sequence
  Synthetic peptide
QASKEYEAVAKFVAA

SEQ ID NO: 27 - protein, 15 aa, Artificial sequence
  Synthetic peptide
AKFVAAWTLKAKTGE

SEQ ID NO: 28 - protein, 15 aa, Artificial sequence
  Synthetic peptide
KAKTGEAADENSEVT

SEQ ID NO: 29 - protein, 15 aa, Artificial sequence
  Synthetic peptide
ENSEVTFRVVPEGVK

SEQ ID NO: 30 - protein, 15 aa, Artificial sequence
  Synthetic peptide
VPEGVKRESYAGAKF

SEQ ID NO: 31 - protein, 15 aa, Artificial sequence
  Synthetic peptide
YAGAKFVAAWTLKAH

SEQ ID NO: 32 - protein, 15 aa, Artificial sequence
  Synthetic peptide
WTLKAHTDKPVYTPD

SEQ ID NO: 33 - protein, 15 aa, Artificial sequence
  Synthetic peptide
PVYTPDQSVKKSDLG

SEQ ID NO: 34 - protein, 15 aa, Artificial sequence
  Synthetic peptide
KKSDLGCGAGGGHDN

SEQ ID NO: 35 - protein, 15 aa, Artificial sequence
  Synthetic peptide
GGGHDNAKFVAAWTL

SEQ ID NO: 36 - protein, 15 aa, Artificial sequence
  Synthetic peptide
VAAWTLKADTLKRPD

SEQ ID NO: 37 - protein, 13 aa, Artificial sequence
  Synthetic peptide
TLKRPDSSVPSSG

SEQ ID NO: 38 - protein, 14 aa, Artificial sequence
  Synthetic peptide
DTLKRPDSSVPSSG

SEQ ID NO: 39 - protein, 15 aa, Mus musculus
ACKPEASYKPSKEES

SEQ ID NO: 40 - protein, 14 aa, Homo sapiens
ISAETRKQTACKPE

SEQ ID NO: 41 - protein, 14 aa, Homo sapiens
ASYKPSREESSSGS

SEQ ID NO: 42 - protein, 14 aa, Homo sapiens
QETSDLDPSKSVTR

SEQ ID NO: 43 - protein, 14 aa, Homo sapiens
NYQLDNGSFKENSQ

SEQ ID NO: 44 - protein, 13 aa, Homo sapiens
NANADDSQENDEP

SEQ ID NO: 45 - protein, 14 aa, Homo sapiens
YSLNDDLKPAKRET

SEQ ID NO: 46 - protein, 14 aa, Homo sapiens
LPEENQAREGYRAI

SEQ ID NO: 47 - protein, 15 aa, Homo sapiens
KTGEAVAEKDSEITF

SEQ ID NO: 48 - protein, 14 aa, Homo sapiens
RVVPEGVKRESYSG

SEQ ID NO: 49 - protein, 14 aa, Homo sapiens
KSDLGCGAGGGLNN

SEQ ID NO: 50 - protein, 13 aa, Homo sapiens
HTDKPVYTPDQSV

SEQ ID NO: 51 - skipped

SEQ ID NO: 52 - protein, 14 aa, Homo sapiens
DNLQHKDSSVPNTG

SEQ ID NO: 53 - protein, 12 aa, Homo sapiens
SAETRKQTACKP

SEQ ID NO: 54 - protein, 20 aa, Homo sapiens
DLTISAETRKQTACKPEIAY

SEQ ID NO: 55 - protein, 12 aa, Homo sapiens
SYKPSREESSSG

SEQ ID NO: 56 - protein, 20 aa, Homo sapiens
VACASYKPSREESSSGSSHA

SEQ ID NO: 57 - protein, 12 aa, Homo sapiens
ETSDLDPSKSVT

SEQ ID NO: 58 - protein, 20 aa, Homo sapiens
DVNQETSDLDPSKSVTRVDD

SEQ ID NO: 59 - protein, 12 aa, Homo sapiens
YQLDNGSFKENS

SEQ ID NO: 60 - protein, 20 aa, Homo sapiens
LVENYQLDNGSFKENSQYQP

SEQ ID NO: 61 - protein, 11 aa, Homo sapiens
ANADDSQENDE

SEQ ID NO: 62 - protein, 19 aa, Homo sapiens
FLTNANADDSQENDEPCKE

SEQ ID NO: 63 - protein, 12 aa, Homo sapiens
SLNDDLKPAKRE

SEQ ID NO: 64 - protein, 20 aa, Homo sapiens
VRVYSLNDDLKPAKRETVLT

SEQ ID NO: 65 - protein, 12 aa, Homo sapiens
PEENQAREGYRA

SEQ ID NO: 66 - protein, 20 aa, Homo sapiens
APDLPEENQAREGYRAIAYS

SEQ ID NO: 67 - protein, 13 aa, Homo sapiens
TGEAVAEKDSEIT

SEQ ID NO: 68 - protein, 21 aa, Homo sapiens
DIYKTGEAVAEKDSEITFIKK

SEQ ID NO: 69 - protein, 12 aa, Homo sapiens
VVPEGVKRESYS

SEQ ID NO: 70 - protein, 20 aa, Homo sapiens
KTLRVVPEGVKRESYSGVTL

SEQ ID NO: 71 - protein, 12 aa, Homo sapiens
SDLGCGAGGGLN

SEQ ID NO: 72 - protein, 20 aa, Homo sapiens
FLEKSDLGCGAGGGLNNANV

SEQ ID NO: 73 - protein, 11 aa, Homo sapiens
TDKPVYTPDQS

SEQ ID NO: 74 - protein, 20 aa, Homo sapiens
LFIHTDKPVYTPDQSVKVRV

SEQ ID NO: 75 - protein, 12 aa, Homo sapiens
NLQHKDSSVPNT

SEQ ID NO: 76 - protein, 20 aa, Homo sapiens
FWKDNLQHKDSSVPNTGTAR